(19) United States
(12) Patent Application Publication
(10) Pub. No.: US 2009/0069426 A1
(43) Pub. Date: Mar. 12, 2009

(54) Methods For Reducing The Amount Of A Proinflammatory Substance In Animal Tissue Or Body Fluid

(75) Inventors: Dale Scott Scherl, Lawrence, KS (US); Ellen Irene Logan, Wamego, KS (US); Kathy Lynn Gross, Topeka, KS (US)

Correspondence Address: COLGATE-PALMOLIVE COMPANY, 909 RIVER ROAD, PISCATAWAY, NJ, 08855, US

(73) Assignee: Hill's Pet Nutrition, Inc., Topeka, KS (US)

(21) Appl. No.: 12/094,188

(22) PCT Filed: Nov. 17, 2006
(86) PCT No.: PCT/US2006/061037

§ 371 Date: Nov. 17, 2008

Related U.S. Application Data
(60) Provisional application No. 60/738,128, filed on Nov. 18, 2005.

(51) Int. Cl. A61K 31/20; C07C 57/02; A61P 29/00

(52) U.S. Cl. 514/560; 554/224

(57) ABSTRACT

A method for reducing the amount of a proinflammatory substance present at an elevated level in animal tissue or body fluid by orally administering to the animal a proinflammatory substance reducing amount of a composition comprising at least one omega-3 fatty acid. The method is useful for reducing inflammation caused by elevated levels of proinflammatory substances including inflammation associated with an oral condition.

### CROSS-REFERENCE TO RELATED APPLICATIONS

This application claims priority to U.S. Provisional Application Ser. No. 60/738.128 filed Nov. 18. 2005, the disclosure of which is incorporated herein by reference.

### BACKGROUND OF THE INVENTION

1. Field of the Invention

The present invention relates generally to methods for reducing the amount of a proinflammatory substance in animal tissue or body fluid.

2. Description of the Related Art

Inflammation and its associated proinflammatory substances are part of an animal's immunological response to such challenges as disease or invading pathogens. But inflammation, which can be internal, external or both, sometimes occurs persistently and at levels that negatively impact the health of the animal. At times, the sustained and/or elevated production of proinflammatory substances may cause inflammation to work against the body's tissues and cause damage.

For example, gingivitis is an inflammatory response involving oral tissues that can occur as a result of irritation or stimulation by resident microorganisms. In a series of events involving several aspects of host immune response, bacterial toxins such as cell wall lipopolysaccharides (LPS) may stimulate defense processes that include production of cytokines, prostanoids, proteases and/or reactive oxygen species. These substances have a function in defense against microbial invaders, but they can also cause collateral hard and soft tissue destruction, ultimately leading to tooth loss. In addition, effects of these substances are not confined to the local environment of the mouth and, therefore, contribute to the load of substances associated with inflammatory processes and tissue destruction systemically.

Nutrition can play an important role in modulation of an animal's immune function. For example, researchers have evaluated the impact that specific fatty acids (FAs) may have on inflammation and proinflammatory substances.

U.S. Patent Application Publication No. 20050043405 discloses a method for restoring a more nearly normal joint function in an osteoarthritic dog. The method comprises feeding to the dog a composition comprising EPA at a concentration of at least about 0.2% by weight. The application asserts that EPA acts to prevent the development of the degenerative process in joint cartilage and thereby improve joint function in osteoarthritic dogs. The application also states that this effect is in addition to an anti-inflammatory action of omega-3 fatty acids, which may be of less importance in canine osteoarthritis because of a limited involvement of inflammation in the osteoarthritis.

Despite ongoing research aimed at understanding inflammation and the role proinflammatory substances play in tissue damage or disease progression, effective management of an inflammatory condition has remained a challenge. Although a number of conventional treatments exist, unfortunately, such treatments have drawbacks including side effects, and may actually be harmful or make the condition worse. For example, steroids can fight inflammation by reducing the production of inflammatory chemicals and are often prescribed for conditions including asthma, inflammatory bowel disease, and inflammatory arthritis. But steroids can have considerable side-effects and are one of the most frequently abused drugs in veterinary and human medicine. There remains, therefore, a need for new or alternative methods and compositions for managing an inflammatory condition, in particular methods and compositions for decreasing levels of proinflammatory substances.

### SUMMARY OF THE INVENTION

The invention provides methods for reducing the amount of a proinflammatory substance present at an elevated level in animal tissue or body fluid by orally administering to the animal a proinflammatory substance reducing amount of a composition comprising at least one omega-3 fatty acid. In one embodiment, the elevated level of proinflammatory substance is associated with an oral condition.

The invention also provides methods for reducing the amount of a proinflammatory substance present at an elevated level in a local biofluid or tissue of an animal by orally administering to the animal a proinflammatory substance reducing amount of a composition comprising at least one omega-3 fatty acid.

The invention further provides methods for preventing, alleviating, or remedying an inflammatory condition secondary to an oral condition in an animal by orally administering to the animal a proinflammatory substance reducing amount of a composition comprising at least one omega-3 fatty acid.

The invention also provides a method for selecting a composition for administration to an animal by making an assessment of presence or absence of an inflammatory condition secondary to an oral condition in the animal and selecting a composition based on the assessment, wherein, if the assessment indicates the presence of an inflammatory condition, the composition selected is one comprising a proinflammatory substance reducing amount of a composition comprising at least one omega-3 fatty acid.

The invention provides articles of manufacture in the form of kits that contain combinations of compositions and other components useful for reducing the amount of a proinflammatory substance present at an elevated level in an animal tissue or body fluid.

Other and further features and advantages of the present invention will be readily apparent to those skilled in the art.

### DETAILED DESCRIPTION OF THE INVENTION

### Definitions

The term “animal” means a human or other animal capable of producing an inflammatory response, including avian, bovine, canine, equine, feline, hicrine, murine, ovine, and porcine animals. Preferably, the animal is a canine or feline.

The term “administering” means causing an animal to ingest a composition, and includes passive administration, i.e., presenting a composition such as a food to an animal for consumption.

The term “proinflammatory substance” herein refers to any substance produced in an animal that is a direct or indirect mediator of inflammation, or is directly or indirectly involved in production of a mediator of inflammation. Non-limiting examples of proinflammatory substances are described below.

An “elevated level” of a proinflammatory substance means a concentration in a tissue or body fluid of an animal that is higher than a normal range for that particular proinflammatory substance in that particular tissue or body fluid. A concentration in the normal range is one that is typically found in a healthy animal. “Reducing” a proinflammatory substance in a tissue or body fluid means lowering the concentration of the substance from a level that is elevated to a level closer to or within the normal range.

The term “blood” means a vascular fluid including cells and other components dispersed therein. A proinflammatory substance “in blood” can be inside or outside cells such as red blood cells and leukocytes, and/or can be associated with a cell membrane, for example.

The term “local biofluid” means a fluid other than blood, in or derived from an animal body. Illustratively, local biofluids include, without limitation, interstitial space fluid, crevicular fluid (e.g., gingival crevicular fluid), sulcus fluid, milk, saliva, tissue fluid, tissue extract, synovial fluid, lymph fluid, mucus, amniotic fluid, vaginal fluid, cerebrospinal fluid, urine, and semen. A local biofluid may comprise cells as well as non-cell components, therefore, a proinflammatory substance “in a local biofluid” can be inside or outside cells and/or associated with a cell membrane.

The term “body fluid” herein is used in its broadest sense, referring to any fluid of an animal regardless of whether the fluid is present inside or outside a blood vessel. Blood and local biofluids, therefore, are examples of “body fluid”.

The term “omega-3 fatty acid” means a member of a group of polyunsaturated fatty carboxylic acids. In general, the omega-3 fatty acids contain 12-26 carbon atoms with methylene-interrupted double bonds, one of which is between the 3rd and 4th carbon atoms as counted from the methyl end of the fatty acid molecule. The physiologically more important omega-3 fatty acids are 18-22 carbons in length and straight chained. Illustratively, omega-3 fatty acids include, without limitation, eicosapentaenoic acid (EPA), docosahexaenoic acid (DHA), α-linolenic acid (ALA), and derivatives thereof. Typically, omega-3 fatty acids are included in food compositions as components of triglycerides. Additional non-limiting examples of derivatives include salts and esters, such as branched or unbranched and/or saturated or unsaturated C₁-C₃₀ alkyl and cycloalkyl esters, in particular C₁-C₆ alkyl esters of omega-3 fatty acids.

The term “oral condition” means any condition affecting the oral cavity that is an etiologic and/or exacerbating factor in inflammation in an animal. Illustratively, oral conditions include, without limitation, dental plaque, gingivitis, periodontitis, and combinations thereof.

### THE INVENTION

In one aspect the present invention provides a method for reducing the amount of a proinflammatory substance present at an elevated level in animal tissue or body fluid. The method comprises orally administering to the animal a proinflammatory substance reducing amount of a composition comprising at least one omega-3 fatty acid. The invention is based upon the discovery that omega-3 fatty acids can reduce local and systemic in vivo levels of proinflammatory substances, particularly when administered systemically, e.g., by mouth as a component of food. Such reduction results in a concomitant reduction in tissue destruction with which these substances are associated. Without being held to a particular theory, it is believed that omega-3 fatty acids can decrease inflammation by providing an alternate substrate for proinflammatory enzymes such as cyclooxygenases, shunting the products of these enzymes to substances less potent in mediating an inflammatory response. For example, production of the proinflammatory mediator prostaglandin E₂ (PGE₂) can shift in favor of the less potent prostaglandin E₃ (PGE₃) when omega-3 fatty acids are substituted for arachidonic acid (AA) as a substrate for cyclooxygenase-1 (COX-1) and cyclooxygenase-2 (COX-2). Similarly, production of the chemoattractant leukotriene B₄ (LTB₄), can shift to the less powerful leukotriene B₅ (LTB₅) when the substrate is shunted from AA to omega-3 fatty acids.

In some embodiments, the invention provides a method of reducing the amount of a proinflammatory substance present at an elevated level in an animal's blood when the elevated level of the proinflammatory substance is associated with an oral condition. The method comprises administering to the animal a proinflammatory substance reducing amount of a composition comprising at least one omega-3 fatty acid.

In a particular embodiment, the animal is a companion animal. A “companion animal” herein is an individual animal of any species kept by a human caregiver as a pet, or any individual animal of a variety of species that have been widely domesticated as pets, including dogs (Canis familiaris) and cats (Felis domesticus), whether or not the individual animal is kept solely or partly for companionship. Thus “companion animals” herein include working dogs, farm cats kept for rodent control, etc., as well as pet dogs and cats. In one embodiment, the animal is a canine. In another embodiment, the animal is a feline.

A variety of proinflammatory substances are known to those skilled in the art. Illustratively, proinflammatory substances include, without limitation, eicosanoids such as, for example, prostaglandins (e.g., PGE₂) and leukotrienes (e.g., LTB₄); gases (e.g., nitric oxide (NO)); enzymes (e.g., phospholipases, inducible nitric oxide synthase (iNOS), COX-1 and COX-2): and cytokines such as, for example, interleukins (e.g., IL-1α, IL-1β, IL-2, IL-3, IL-4, IL-5, IL-6, IL-8, IL-10, IL-12 and IL-18), members of the tumor necrosis factor family (e.g., TNF-α, TNF-β and lymphotoxin β), interferons (e.g., IFN-β and IFN-γ), granulocyte/macrophage colony-stimulating factor (GM-CSF), transforming growth factors (e.g., TGF-β1, TGF-β2 and TGF-β3, leukemia inhibitory factor (LIF), ciliary neurotrophic factor (CNTF), migration inhibitory factor (MIF), monocyte chemoattractant protein (MCP-1). macrophage inflammatory proteins (e.g., MIP-1α, MIP-1β and MIP-2), and RANTES. In one embodiment, the proinflammatory substance is a cytokine or an enzyme. In another embodiment, the proinflammatory substance is selected from the group consisting of IL-6, iNOS, and COX-2.

The present invention is not limited to any particular source of omega-3 fatty acids. Illustratively, sources of omega-3 fatty acids include, without limitation, fish (e.g., menhaden, sardine, herring, tuna, salmon), fish oil, fish meal, plant oil, algae, algae oil, flax seed, flax seed oil, canola, canola oil, soybean, soybean oil, walnut, walnut oil, and mixtures thereof. An omega-3 fatty acid also can be obtained by chemical synthesis. An omega-3 fatty acid can be incorporated into preparations in the form of the free acid or as a pharmaceutically or nutritionally acceptable salt. The at least one omega-3 fatty acid, can be in a highly purified, substantially purified, partially purified, or non-purified form. Illustrative omega-3 fatty acid purification methods are disclosed in the following references: U.S. Pat. No. 4,377.526. U.S. Pat. No. 4,792,418 and references disclosed therein, and U.S. Patent Application Publication No. 2004/0236128 and references disclosed therein.

The omega-3 fatty acid content of any source, such as fish oil, for example, can be quantitated using techniques known to the skilled artisan or by a commercial laboratory. In one embodiment, the at least one omega-3 fatty acid is derived from a source selected from the group consisting of fish oil, plantoil, and combinations thereof. In a composition useful according to the invention, the source of the at least one omega-3 fatty acid can be included as an ingredient, i.e., without extraction or purification of the omega-3 fatty acid component from the source.

In another aspect, the present invention provides a method for reducing the amount of a proinflammatory substance present at an elevated level in animal tissue or body. The method comprises administering to the animal a composition comprising at least one omega-3 fatty acid but that is essentially free of one or more other omega-3 fatty acids. For example, the composition comprises EPA and is essentially free of DHA and/or ALA. An “essentially free” amount of one or more omega-3 fatty acids is intended to mean that these specific omega-3 fatty acid(s) are substantially absent in the composition or are present in an amount that is ineffective by itself to reduce a proinflammatory substance present at an elevated level in a tissue or body fluid of an animal. A food composition as described herein, for example, can be essentially free of one or more omega-3 fatty acids, the total essentially free amount being a quantity of less than about 0.1%, less than about 0.03%, less than about 0.01%, less than about 0.003%, or less than about 0.001% by weight on a dry matter (DM) basis.

Illustratively, a composition that comprises at least one omega-3 fatty acid can be a food composition, a supplement, a treat or a toy, it being noted that some, but not all, supplements, treats and toys are themselves food compositions. A composition that comprises at least one omega-3 fatty acid can be a wet or dry composition (e.g., a wet or dry food composition) and the at least one omega-3 fatty acid can be either incorporated therein or on the surface of any composition component, such as, for example, by spraying, agglomerating, dusting, or precipitating on the surface.

Food compositions are typically administered to an animal by feeding. Where the animal is a companion animal, a food composition useful in the method of the invention is typically one that is nutritionally and/or organoleptically adapted for feeding to such an animal. A food composition so adapted is referred to herein as a “pet food”. Pet foods can be more particularly adapted to the special nutritional needs of canines or felines, or to certain subpopulations thereof such as large-breed dogs, adult dogs or cats, senior dogs or cats, geriatric dogs or cats, etc.

A food composition of an animal can meet its ordinary nutritional requirements, which a skilled artisan can determine based upon the animal's species, age, sex, weight, and other factors. In some embodiments, a food composition that comprises at least one omega-3 fatty acid provides a substantially nutritionally complete food for the intended recipient animal. A “nutritionally complete food” is a food that includes sufficient nutrients for maintenance of normal health of a healthy animal if the food provides substantially all of the animal's diet.

In the pet food industry, for example, foods are generally classified as “wet” or “dry”. A wet food has a relatively high amount of water and is usually present in a can or a container wherein air is substantially or totally excluded. Examples of such foods are “chunk and gravy”, individual solid particles in the presence of liquid gravy or a loaf type material which generally takes the shape of the receptacle. A dry food is generally a baked or extruded material, the latter then cut into individual shaped portions, usually known as kibbles. The at least one omega-3 fatty acid can be readily incorporated into a wet food through conventional means. Encapsulation can be employed to protect the at least one omega-3 fatty acid from air oxidation in a dry food. Additionally, antioxidants and nitrogen sweeps of packaging can be employed. This is exemplified by U.S. Pat. No. 4,895,725 which has special emphasis on the micro-encapsulation of specific fish oils.

Illustratively, a typical food for a canine of 1-6 years of age contains on a dry matter basis about 23% protein, about 15% fat, about 0.6% phosphorus, 0.6% calcium and about 0.3% sodium; and, for older canines and felines, a typical food can be, for example, as provided in Table 1.

TABLE 1
Typical Composition of Diet for Older Canines and Felines

| Component | Canine | Feline |
|---|---|---|
| crude protein (% dry matter) | 15-25 | 26-50 |
| crude fat (% dry matter) | 7-20 | 10-30 |
| crude fiber (% dry matter) | >2 | <10 |
| calcium (% dry matter) | 0.5-1.2 | 0.6-1.5 |
| phosphorus (% dry matter) | 0.25-1.2 | 0.5-1.5 |
| sodium (% dry matter) | 0.15-0.5 | 0.15-0.5 |
| magnesium (% dry matter) | 0.05-0.2 | 0.05-0.15 |

In another embodiment, a composition comprises at least one omega-3 fatty acid is in the form of a supplement. Supplements include, for example, a feed or food used with another feed or food to improve the nutritive balance or performance of the total. Supplements can include compositions that are fed undiluted as a supplement to other feeds or foods, offered free choice with other parts of an animal's ration that are separately available, or diluted and mixed with an animal's regular feed or food to produce a complete feed or food. Supplements can be in various forms including, for example, powders, liquids (including gels), syrups, pills, encapsulated compositions, etc.

In a further embodiment, a composition comprises at least one omega-3 fatty acid in the form of a treat. Illustratively, treats for canines include, for example, dog biscuits in the shape of dog bones. Treats can be nutritional, wherein the composition comprises one or more nutrients, and can, for example, have a composition as described above for food, or can be substantially non-nutritional except for their omega-3 fatty acid content. The at least one omega-3 fatty acid can be coated onto the treat, incorporated into the treat, or both.

In a yet further embodiment, a composition comprises at least one omega-3 fatty acid in the form of a toy. Toys include, for example, chewable toys. Illustratively, toys for canines include, for example, artificial bones. The at least one omega-3 fatty acid, for example, can be present in a coating on the surface of a toy or on the surface of a component of the toy, or can be incorporated partially or fully throughout the toy, or both. Illustrative toys suitable for modification in accordance with the present invention are disclosed in the following references: U.S. Pat. No. 5,339,771 and references disclosed therein and U.S. Pat. No. 5,419,283 and references disclosed therein.

The term “toys” means both partially consumable toys (e.g., toys comprising plastic components) and fully consumable toys (e.g., rawhides and various artificial bones). It should be further recognized that toys can be for either human or non-human use, particularly for companion, farm, and zoo animal use, and particularly for dog or cat use. The terms “treat” and “toy” can be considered interchangeable for the purposes of this specification. However, in general a treat is fully edible and a toy in accordance with the invention has an edible coating.

At least one omega-3 fatty acid can be present in a nutritional food per se or in a snack, supplement, treat, toy, etc. It can also be present in the liquid portion of a composition such as water or another fluid. If desired the omega-3 fatty acid can be orally administered in a nutraceutical or pharmaceutical dosage form such as a capsule, tablet, caplet, syringe, and the like. Within a dosage form, the at least one omega-3 fatty acid can be present as a powder or a liquid such as a gel. Any of the usual nutraceutical or pharmaceutical carriers can be employed such as water, glucose, sucrose and the like together with the omega-3 fatty acids.

The composition is administered at a frequency and for a period effective to reduce a proinflammatory substance present at an elevated level. Typically and most conveniently, the composition is administered at least once daily, but in certain situations less frequent, e.g., twice weekly or weekly, administration can be effective. For greatest benefit, administration should continue for at least about 1 week, for example at least about 2 weeks, at least about 3 weeks, at least about 1 month, at least about 2 months, at least about 3 months, at least about 4 months, at least about 5 months, at least about 6 months, at least about 1 year, at least about 2 years, or at least about 3 years. In one embodiment, administration continues from a time of initiation for substantially the remainder of the animal's life.

The time of initiation can be at any stage of the animal's life (i.e., there is no upper or lower age limit for initiating administration). For example, in the case of canine and feline companion animals, administration can be initiated when the animal is at least about 0.25, at least about 0.5. at least about 0.75, at least about 1, at least about 2, at least about 3, at least about 4, at least about 5, at least about 6, at least about 7, at least about 8, at least about 9, or at least about 10 years old. In one embodiment, administration is initiated at or near birth.

In some embodiments, a single composition is administered to an animal for the entire period of administration. In other embodiments, different compositions that comprise at least one omega-3 fatty acid are administered to the animal at different times. For example, the selection and/or amounts of individual omega-3 fatty acids can, if desired, vary over the period of administration, or administration can switch from a wet to a dry food, or vice versa, at any time or repeatedly.

The dosages of omega-3 fatty acids can be adjusted on a body weight basis and may thus be adapted to be suitable for any animal regardless of its size. For example, a 20 kg dog can be expected to consume about 275 g DM of food per day. Thus, administration of a food composition that comprises a total omega-3 fatty acid amount of at least about 0.1% DM by weight, illustratively about 0.1% to about 20%, about 0.3% to about 17%, about 0.4% to about 14%, about 0.5% to about 11%, about 0.6% to about 8%, about 0.7% to about 5%, about 0.8% to about 2%, or about 0.9% to about 1% DM by weight would amount to administering to the dog a total omega-3 fatty acid amount of at least about 1.4 g/kg body weight, illustratively about 1.4 to about 275, about 4.1 to about 234, about 5.5 to about 193, about 6.9 to about 151, about 8.3 to about 110, about 9.6 to about 69, about 11 to about 27.5, or about 12.4 to about 13.6 g/kg body weight, respectively.

In one embodiment, a total omega-3 fatty acid amount of at least about 0.1% by weight on a DM basis is present in the food composition administered by the method of the invention. Illustratively, the total omega-3 fatty acid amount can be provided as part of the usual nutrient food ration on a daily basis or the same daily quantity can be provided to the animal in a treat or supplement. Additionally, a combination of these methods or any other dosing can be employed as long as the effective quantity of omega-3 fatty acid is provided. In one embodiment, the at least one omega-3 fatty acid is selected from the group consisting of EPA, DHA, and combinations thereof.

In further embodiments, the present invention provides a method for reducing the amount of a proinflammatory substance present at an elevated level in a local biofluid or tissue of an animal comprising administering to the animal a proinflammatory substance reducing amount of a composition comprising at least one omega-3 fatty acid.

In one embodiment, the animal is a canine or feline. In another embodiment, the animal is a companion animal.

In one embodiment, the proinflammatory substance is present at an elevated level in a local tissue, e.g., gingival tissue. In another embodiment, the proinflammatory substance is present at an elevated level in a local biofluid, e.g., crevicular fluid.

Without being held to a particular theory, it is believed that the effects of a proinflammatory substance are not confined to a local environment and, therefore, contribute to the total systemic load of substances associated with inflammatory processes and tissue destruction. Attenuation of a level of a proinflammatory substance can result in a decrease in the local and systemic load of proinflammatory substances thereby affecting an inflammatory condition of an animal.

An inflammatory condition can be acute or chronic. The term “inflammatory condition” herein not only refers to an inflammatory condition, disorder, or disease per se, but also to any condition, disorder, or disease that develops or progresses as a result of an inflammatory condition. Illustratively, inflammatory conditions include, without limitation, gingivitis, periodontitis, rheumatoid arthritis, bursitis, osteoarthritis, systemic lupus, asthma, hepatitis, bronchitis, acute gouty arthritis, psoriatic arthritis, colitis, Crohn's disease, an allergic condition (e.g., bronchial asthma, allergic rhinitis, drug-induced dermatitis, contact and atopic dermatitis), a chronic skin condition (e.g., dermatitis herpetiformis, pemphigus, severe psoriasis and severe seborrheic dermatitis, chronic allergic and inflammatory conditions of the uvea, iris, conjunctiva and optic nerves of the eyes, an acute coronary syndrome (e.g., unstable angina, acute myocardial infarction, sudden cardiac death, coronary plaque rupture, thrombosis), inflammatory bowel disease, and combinations thereof. An inflammatory condition, particularly a chronic condition, also can contribute to or be a risk factor for the development or progression of other conditions, disorders, or diseases, including, without limitation, cancer, cachexia, cardiovascular disease, diabetes, osteoporosis, and neurodegenerative disorders such as Alzheimer's disease.

In one aspect, the present invention provides a method for preventing an inflammatory condition in an animal comprising orally administering to the animal an inflammatory condition preventing amount of a composition comprising at least one omega-3 fatty acid. The term “preventing” an inflammatory condition herein refers to preventing or decreasing the likelihood of developing a condition.

In other embodiments, the present invention provides a alleviating or remedying an inflammatory condition of an animal, the method comprising administering to the animal a composition comprising at least one omega-3 fatty acid in a total omega-3 fatty acid amount effective to decrease a level of a proinflammatory substance in a body fluid. The phrase “alleviating or remedying” herein is inclusive of reducing severity of or eliminating an inflammatory condition.

In further embodiments, the present invention provides a method for selecting a composition for administration to an animal comprising making an assessment of presence or absence of an inflammatory condition secondary to an oral condition in the animal and selecting a composition based on the assessment; wherein if the assessment indicates the presence of an inflammatory condition, the composition selected is one comprising a proinflammatory substance reducing amount of a composition comprising at least one omega-3 fatty acid.

In one embodiment, assessing comprises determining whether the animal has symptoms of such an inflammatory condition. In another embodiment, assessing comprises determining a level of a proinflammatory substance in a tissue or body fluid of an animal. For example, the level can be determined using a body fluid sample taken from the animal. Illustratively, a blood sample can be drawn from an animal using a syringe and the level of a proinflammatory substance determined in the blood or in serum from the sample.

A level of a proinflammatory substance can be determined in a body fluid sample using standard assays known in the art. Typically, an assay can be chosen based on the type of proinflammatory substance being determined as well as the assay's suitability for quantifying the level of the substance in a particular sample. For example, a commercially available immunoassay utilizing monoclonal antibodies reactive to one or more epitopes on polypeptides or a competitive binding assay can be used for determining the serum level of a proinflammatory substance that is a protein. Alternatively, the level of such a proinflammatory substance may be determined by quantifying the level of its mRNA in cells that express the mRNA and which are present in the body fluid sample. Alternatively, the level of a proinflammatory substance can be determined by measuring activity level of the substance.

In some embodiments, a level is determined using one or more assays independently selected from the group consisting of enzyme immunoassays (EIAs), enzyme-linked immunosorbent assays (ELISAs), immunofluorescent assays (IFAs), radioimmunoassays (RIAs), western blot assays, northern blots, biochemical assays, enzymatic assays, and colorimetric assays. A variety of labels and conjugation techniques are known by those skilled in the art and can be used in the various biochemical, nucleic acid and amino acid assays.

A level of a proinflammatory substance can be an “observed” level that is compared to a reference level for the particular proinflammatory substance. For example, a reference level can be determined in a reference animal known not to have an inflammatory condition. A reference animal (i.e., the animal used to determine a reference level of a proinflammatory substance) will generally be of the same species, optionally of the same breed and/or of about the same age, as the animal for which the observed level is obtained.

In some embodiments, a tissue or body fluid sample is collected at a point of care facility, for example, a veterinary clinic, and an observed level of a proinflammatory substance is determined at the point of care facility. The term “point of care facility” herein refers to a place where an animal can be seen by a health care practitioner (e.g., medical doctor, veterinarian, medical assistant, physician's assistant, nurse, etc.) for evaluation and diagnosis. Non-limiting examples of a point of care facility include a hospital, office of a physician or veterinarian, veterinarian's clinical office, animal's home, farm, stable, and barracks where the animal is kept.

Illustratively, a health care practitioner (e.g., doctor or veterinarian) or the animal's caretaker can obtain a sample from an animal; determine an observed level of a proinflammatory substance (e.g., by applying the sample to a kit); read the results on-site; and determine the level of the substance; or, optionally, send the sample to a secondary facility. The term “secondary facility” herein refers to a laboratory such as a commercial testing laboratory where clinical samples are evaluated. For example, rather than determining an observed level of the proinflammatory substance oneself, one can obtain the results performed by others.

In some embodiments, a sample is collected at a point of care facility and sent to a secondary facility for determining an observed level of a proinflammatory substance. In one particular embodiment, a secondary facility is located off site from a point of care facility. The term “off site” herein refers to a physical location remote from the point of care facility. In another embodiment, a secondary facility is located at a facility directed by a business organization other than the organization directing the point of care facility.

In some embodiments, comparing the observed level to a reference level is performed by a point of care facility or a secondary facility. In some embodiments, the assessing is performed by a point of care facility. For example, a health care practitioner (e.g., doctor or veterinarian) or the animal's caretaker can assess the animal and select a composition (if necessary).

In a further aspect, the present invention provides kits suitable for administering an omega-3 fatty acid to an animal. The kits comprise in separate containers in a single package or in separate containers in a virtual package, as appropriate, at least one omega-3 fatty acid and at least one of (1) one or more ingredients suitable for consumption by an animal, (2) instructions for how to combine the omega-3 fatty acid and other kit components to produce a composition useful for reducing the amount of a proinflammatory substance present at an elevated level in animal tissue or body fluid, and (3) instructions for how to use the omega-3 fatty acid and other components of the present invention, particularly for the benefit of the animal. When the kit comprises a virtual package, the kit is limited to instructions in a virtual environment in combination with one or more physical kit components. The kit contains the omega-3 fatty acid and other components in amounts sufficient to produce a composition useful for reducing the amount of a proinflammatory substance present at an elevated level in animal tissue or body fluid. Typically, the omega-3 fatty acid and the other suitable kit components are admixed just before consumption by an animal. In one embodiment, the kit contains a packet containing one or more omega-3 fatty acid and a container of food for consumption by an animal. The kit may contain additional items such as a device for mixing the omega-3 fatty acid and ingredients or a device for containing the admixture, e.g., a food bowl. In another embodiment, the omega-3 fatty acid is mixed with additional nutritional supplements such as vitamins and minerals that promote good health in an animal.

In another aspect, the present invention provides a means for communicating information about or instructions for one or more of (1) using omega-3 fatty acid for reducing the amount of a proinflammatory substance present at an elevated level in animal tissue or body fluid, (2) admixing omega-3 fatty acid with the other components of the present invention, (3) administering omega-3 fatty acid to an animal, alone or in combination with the other elements of the present invention, and (4) using the kits of the present invention for reducing the amount of a proinflammatory substance present at an elevated level in animal tissue or body fluid, the means comprising a document, digital storage media, optical storage media, audio presentation, or visual display containing the information or instructions. In certain embodiments, the communicating means comprises a document, digital storage media, optical storage media, audio presentation, or visual display containing the information or instructions. Preferably, the communication means is a displayed web site or a brochure, product label, package insert, advertisement, or visual display containing such information or instructions. Useful information includes one or more of (1) methods and techniques for combining and administering the omega-3 fatty acid and/or other components and (2) contact information for animals or their caregivers to use if they have a question about the invention and its use. Useful instructions include amounts for mixing and administration amounts and frequency. The communication means is useful for instructing on the benefits of using the present invention and communicating the approved methods for administering the invention to an animal.

In a further aspect, the present invention provides for a use of a composition of the present invention to prepare a medicament. In another, the invention provides for the use of such composition to prepare a medicament for maintaining and/or improving animal health, e.g., for reducing the amount of a proinflammatory substance present at an elevated level in animal tissue or body fluid. Generally, medicaments are prepared by admixing a compound or composition with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to an animal.

The invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise. Similarly, the words “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents. patent applications, and publications mentioned herein are incorporated herein by reference to the extent allowed by law for the purpose of describing and disclosing the compounds, processes, techniques, procedures, technology, articles, and other compositions and methods disclosed therein that might be used with the present invention. However, nothing herein is to be construed as an admission that the invention is not entitled to antedate such disclosure by virtue of prior invention.

### EXAMPLES

The invention can be further illustrated by the following examples of preferred embodiments thereof, although it will be understood that these examples are included merely for purposes of illustration and are not intended to limit the scope of the invention unless otherwise specifically indicated.

### Example 1

This example illustrates that a food composition enriched with at least one omega-3 fatty acid decreases in vivo local and systemic levels of proinflammatory substances in a body fluid of an animal.

Forty (40) beagle dogs were randomly assigned to two groups (i.e., control and test group) and their gingival tissues were brought to a state of optimal health (prophylaxis, daily tooth brushing, daily chlorhexidine swabbing). During a one-week period, the control group received a food containing <0.01% EPA and up to 0.01% DHA by weight on a dry matter basis. The second group (test group) received the same food as the control group but enriched with 0.48% to 0.59% EPA and 0.5% to 0.61% DHA by weight. The dogs continued to receive their assigned foods for the remainder of the 12 weeks.

After this one-week period, dogs were again subjected to a prophylaxis, and then monitored for 12 weeks for parameters associated with inflammation, and specifically gingivitis. These parameters were plaque, gingivitis, crevicular fluid (CF) volume, CF PGE₂, CF LTB₄, CF C-reactive protein (CRP), sulcus IL-1β mRNA, sulcus IL-6 mRNA, sulcus TNFα mRNA, sulcus iNOS mRNA, sulcus COX-2 mRNA, serum PGE₂, serum LTB₄, serum CRP, blood IL-1β mRNA, blood IL-6 mRNA, blood TNFα mRNA, blood iNOS mRNA, and blood COX-2 mRNA. Plaque was included as a reference measure to enable a determination that any reduction in inflammation or proinflammatory substances was due to the omega-3 fatty acids and not simply to a reduction in plaque. Methods used to analyze for mRNA and any particular molecules were routine methods known to skilled artisans. The results are shown in Table 1.

TABLE 1
Group Averages

| | | Day 0 | Day 16 | Day 28 | Day 44 | Day 56 | Day 70 | Day 84 |
|---|---|---|---|---|---|---|---|---|
| Plaque | Control | 0.00 | 9.82 | 9.14 | 9.02 | 9.57 | 9.71 | 10.18 |
| | Omega-3 | 0.00 | 10.46 | 9.05 | 9.53 | 9.76 | 10.31 | 10.51 |
| | fatty acid | | | | | | | |
| Gingivitis | Control | 0.46 | 0.53 | 0.67 | 0.77 | 0.84 | 0.90 | 0.87 |
| | Omega-3 | 0.52 | 0.61 | 0.70 | 0.79 | 0.89 | 0.93 | 0.94 |
| | fatty acid | | | | | | | |
| CF vol | Control | 0.48 | 0.80 | 0.83 | 0.74 | 0.88 | 0.85 | 0.94 |
| | Omega-3 | 0.42 | 0.69 | 0.78 | 0.63 | 0.85 | 0.82 | 0.90 |
| | fatty acid | | | | | | | |

Crevicular Fluid (normalized)

| | | | | | | | | |
|---|---|---|---|---|---|---|---|---|
| PGE2 (pg/ml) | Control | 364 | 246 | 186 | 272 | 149 | 198 | 230 |
| | Omega-3 | 356 | 164 | 115 | 123 | 95 | 85 | 108 |
| | fatty acid | | | | | | | |
| LTB4 (pg/ml) | Control | 84 | 91 | ND | 34 | 15 | 68 | 94 |
| | Omega-3 | 163 | 174 | ND | 42 | 32 | 66 | 79 |
| | fatty acid | | | | | | | |
| CRP (ng/ml) | Control | 7.72 | 3.67 | 3.88 | 4.56 | 3.09 | 3.44 | 2.92 |
| | Omega-3 | 5.38 | 4.19 | 3.34 | 4.24 | 3.11 | 3.37 | 2.93 |
| | fatty acid | | | | | | | |

Sulcus Cells (increase above control)

| | | | | | | | | |
|---|---|---|---|---|---|---|---|---|
| I1-1B | Control | 0.07 | 0.95 | −0.60 | ND | 1.54 | 2.14 | 2.19 |
| | Omega-3 | −3.74 | 2.91 | −3.60 | ND | 1.80 | 6.68 | 6.42 |
| | fatty acid | | | | | | | |
| IL-6 | Control | ND | ND | ND | ND | −7.89 | −22.98 | −14.89 |
| | Omega-3 | 1.00 | ND | −6.33 | 8.28 | −4.38 | −40.36 | −213 |
| | fatty acid | | | | | | | |
| TNF | Control | −0.08 | −2.76 | −6.43 | ND | −5.62 | −3.49 | −5.42 |
| | Omega-3 | −4.33 | 1.73 | −4.33 | ND | −0.33 | −2.0 | −2.1 |
| | fatty acid | | | | | | | |
| INOS | Control | −3.94 | −2.24 | −2.88 | ND | −3.72 | −21.5 | −27.1 |
| | Omega-3 | 1.03 | 1.16 | −1.91 | ND | −1.60 | −11.8 | −10.4 |
| | fatty acid | | | | | | | |
| COX-2 | Control | 0.34 | 2.23 | 0.20 | ND | 0.21 | 1.52 | 7.06 |
| | Omega-3 | 5.75 | 4.96 | 1.02 | ND | 1.88 | −0.16 | 3.29 |
| | fatty acid | | | | | | | |

Whole Blood (increase above control)

| | | | | | | | | |
|---|---|---|---|---|---|---|---|---|
| I1-1B | Control | 0.06 | −0.12 | ND | 0.44 | −1.08 | 1.43 | 1.07 |
| | Omega-3 | −3.28 | 1.44 | 2.55 | 3.53 | −3.36 | −0.87 | −0.14 |
| | fatty acid | | | | | | | |
| IL-6 | Control | 0.01 | 225 | ND | 3.72 | ND | 1.50 | 2.61 |
| | Omega-3 | −1.04 | 69.6 | 16.1 | 5.97 | −2.75 | ND | ND |
| | fatty acid | | | | | | | |
| TNF | Control | 0.09 | −1.73 | 1.04 | −8.49 | −4.79 | −6.46 | −3.43 |
| | Omega-3 | −0.14 | −5.71 | 0.08 | −6.78 | −8.07 | −4.66 | −5.82 |
| | fatty acid | | | | | | | |
| iNOS | Control | 0.00 | 17.49 | ND | −2.63 | −4.64 | 1.24 | −0.08 |
| | Omega-3 | 1.51 | 7.63 | ND | −2.81 | 1.58 | ND | ND |
| | fatty acid | | | | | | | |
| COX-2 | Control | −0.01 | 23.16 | ND | 2.35 | −6.22 | 0.08 | 0.73 |
| | Omega-3 | −2.39 | 6.79 | 11.64 | 4.65 | −3.16 | 0.81 | −0.92 |
| | fatty acid | | | | | | | |

Serum

| | | | | | | | | |
|---|---|---|---|---|---|---|---|---|
| PGE2 (pg/ml) | Control | 162 | 189 | 133 | 112 | 108 | 114 | 114 |
| | Omega-3 | 135 | 111 | 78.7 | 87.6 | 98.2 | 91.4 | 113 |
| | fatty acid | | | | | | | |
| LTB4 (pg/ml) | Control | 85.4 | 466 | 357 | 86.9 | 149 | 287 | 622 |
| | Omega-3 | 105 | 448 | 283 | 77.4 | 159 | 267 | 595 |
| | fatty acid | | | | | | | |
| CRP (ng/ml) | Control | 3502 | 3025 | 3053 | 3157 | 2415 | 4277 | 2829 |
| | Omega-3 | 2751 | 2167 | 1415 | 3265 | 1703 | 2597 | 1629 |
| | fatty acid | | | | | | | |

As shown in Table 1, there was no difference in plaque accumulation between the control group and the test group. This result was expected and confirmed the validity of the control. Further, there was no difference in gingivitis score or CF volume between the two groups.

As shown in Table 1, CF PGE₂ levels (normalized to CF volume) were reduced to a greater extent in dogs fed the omega-3 fatty acid enriched food than in dogs fed the control food. At the end of the study there was a 38% reduction in CF PGE₂ levels (from baseline) for the control group, while the test group had a 68% reduction from baseline. Additionally, at the end of the study, animals fed the omega-3 fatty acid enriched food had an average of 52% less CF PGE₂ than the control animals.

Further, as shown in Table 1, CF LTB₄ levels (normalized to CF volume) over time were not different in control animals as compared to animals fed the omega-3 fatty acid enriched food. This profile is mirrored in the serum LTB₄ levels. Further, CF CRP levels (normalized to CF volume) were not different in control animals and animals fed omega-3 fatty acid enriched food. Note that this profile is mirrored in the serum CRP levels.

However. as compared to the control group, dogs fed an omega-3 fatty acid-enriched food had decreased serum IL-6 levels at day 16 of the study. Animals in the control group had an average of 225-fold higher IL-6 mRNA than baseline, and the animals fed omega-3 fatty acid enriched foods had an average of 70-fold higher IL-6 mRNA than baseline, or a 69% difference. IL-6 mRNA levels collected from the cells residing in the sulcus did not show a similar response.

Further, animals fed the omega-3 fatty acid enriched food had directionally decreased levels of serum iNOS over time. Animals fed control food had an average of 17-fold higher iNOS mRNA than baseline, and the animals fed the omega-3 fatty acid enriched foods had an average of 8-fold higher iNOS mRNA than baseline, or 56% difference. Conversely, iNOS mRNA levels collected from the cells residing in the sulcus did not show a similar response.

With respect to serum COX-2 levels over time, the results showed that at day 16 of the study, the food containing omega-3 fatty acid reduced the amount of COX-2. Animals fed control food had an average of 23-fold higher COX-2 mRNA than baseline, and the animals fed the omega-3 fatty acid-enriched food had an average of 7-fold higher COX-2 mRNA than baseline, or 71% difference. COX-2 mRNA levels collected from the cells residing in the sulcus did not show a similar response.

These results demonstrate that local and systemic levels of proinflammatory substances are reduced in animals fed an omega-3 fatty acid enriched food composition.

Methods described herein utilize laboratory techniques well known to skilled artisans and can be found in laboratory manuals such as Sambrook, J., et al., Molecular Cloning: A Laboratory Manual, 3rd ed. Cold Spring Harbor Laboratory Press, Cold Spring Harbor, N.Y. (2001); Spector. D. L. et al. Cells: A Laboratory Manual, Cold Spring Harbor Laboratory Press. Cold Spring Harbor, N.Y. (1998), and Hampton R. et al., Serological Methods: A Laboratory Manual, APS Press, St Paul, Minn. (1990).

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

What is claimed is:

1. A method for reducing the amount of a proinflammatory substance present at an elevated level in animal tissue or body fluid comprising orally administering to the animal a proinflammatory substance reducing amount of a composition comprising at least one omega-3 fatty acid.

2. The method of claim 1 wherein the composition comprises at least about 0.1% omega-3 fatty acid by weight on a dry matter basis.

3. The method of claim 1 wherein the elevated level is associated with an oral condition.

4. The method of claim 3 wherein the oral condition comprises dental plaque deposit.

5. The method of claim 1 wherein the animal is an animal of the order Carnivora.

6. The method of claim 1 wherein the animal is canine or feline.

7. The method of claim 1 wherein the animal is a companion animal.

8. The method of claim 1 wherein the proinflammatory substance is a cytokine or an enzyme.

9. The method of claim 1 wherein the proinflammatory substance is selected from the group consisting of IL-6, iNOS, and COX-2.

10. The method of claim 1 wherein the omega-3 fatty acid is selected from the group consisting of ALA, EPA, DHA, and combinations thereof.

11. The method of claim 1 wherein the composition is in the form of a food, a treat, a supplement, or a toy.

12. The method of claim 11 wherein the composition is a food comprising about 0.1% to about 20% total omega-3 fatty acids by weight on a dry matter basis.

13. A method for reducing the amount of a proinflammatory substance present at an elevated level in a local biofluid or tissue of an animal comprising orally administering to the animal a proinflammatory substance reducing amount of a composition comprising at least one omega-3 fatty acid.

14. The method of claim 13 wherein the composition comprises at least about 0.1% omega-3 fatty acid by weight on a dry matter basis.

15. The method of claim 13 wherein the animal is canine or feline.

16. The method of claim 13 wherein the animal is a companion animal.

17. The method of claim 13 wherein the proinflammatory substance is present at an elevated level in gingival tissue.

18. The method of claim 13 wherein the proinflammatory substance is present at an elevated level in crevicular fluid.

19. A method for preventing, alleviating, or remedying an inflammatory condition secondary to an oral condition of in animal comprising orally administering to the animal a proinflammatory substance reducing amount of a composition comprising at least one omega-3 fatty acid.

20. The method of claim 19 wherein the composition comprises at least about 0.1% omega-3 fatty acid by weight on a dry matter basis.

21. A method for selecting a composition for administration to an animal comprising:
making an assessment of presence or absence of an inflammatory condition secondary to an oral condition in the animal; and
selecting a composition based on said assessment,
wherein, if the assessment indicates the presence of an inflammatory condition, the composition selected is one comprising a proinflammatory substance reducing amount of a composition comprising at least one omega-3 fatty acid.

22. A kit suitable for administering omega-3 fatty acid to an animal comprising in separate containers in a single package or in separate containers in a virtual package, as appropriate, at least one omega-3 fatty acid and at least one of (1) one or more ingredients suitable for consumption by an animal, (2) instructions for how to combine the omega-3 fatty acid and other kit components to produce a composition useful for reducing the amount of a proinflammatory substance present at an elevated level in animal tissue or body fluid, and (3) instructions for how to use the omega-3 fatty acid and other components of the present invention.

23. A means for communicating information about or instructions for one or more of (1) using omega-3 fatty acid reducing the amount of a proinflammatory substance present at an elevated level in animal tissue or body fluid, (2) admixing omega-3 fatty acid with the other components of the present invention, (3) administering omega-3 fatty acid to an animal, alone or in combination with the other elements of the present invention, and (4) using the kits of the present invention for reducing the amount of a proinflammatory substance present at an elevated level in animal tissue or body fluid, the means comprising a document, digital storage media, optical storage media, audio presentation, or visual display containing the information or instructions.

24. The means of claim 23 selected from the group consisting of a displayed web site, brochure, product label, package insert, advertisement, or visual display.

25. A use of a composition that comprises at least one omega-3 fatty acids to prepare a medicament for reducing the amount of a proinflammatory substance present at an elevated level in animal tissue or body fluid.
